(19) United States
(12) Patent Application Publication
(10) Pub. No.: US 2010/0022501 A1
(43) Pub. Date: Jan. 28, 2010

(54) INJECTABLE OR ORALLY DELIVERABLE FORMULATIONS OF AZETIDINE DERIVATIVES

(75) Inventors: Maria-Teresa PERACCHIA, Paris (FR); Gilbert GAUDEL, Paris (FR); Sophie COTE, Antony (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMA SA, Antony (FR)

(21) Appl. No.: 12/573,465

(22) Filed: Oct. 5, 2009

Related U.S. Application Data
(63) Continuation of application No. 11/754,569, filed on May 29, 2007.
Application No. 11/754,569 is a continuation of application No. PCT/FR2005/003263, filed on Dec. 23, 2005.

(30) Foreign Application Priority Data
Dec. 27, 2004 (FR) 0413937

(51) Int. Cl. A61K 31/397; A61P 43/00

(52) U.S. Cl. 514/210.01

(57) ABSTRACT

The invention concerns injectable or orally deliverable binary or ternary formulations of azetidine derivatives. The azetidine derivatives used in the inventive pharmaceutical compositions can be represented by the general formulae (Ia) or (Ib), wherein Ar is an aromatic or heteroaromatic group optionally substituted by one or more among (C₁-C₄)alkyl, halogen, NO₂, CN, (C₁-C₄) alkoxy or OH.

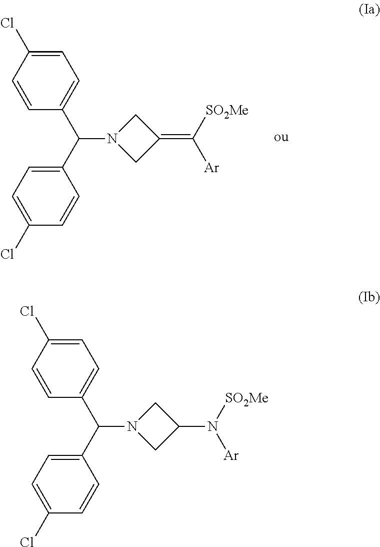

### CROSS REFERENCE TO RELATED APPLICATIONS

This application is a continuation of U.S. application Ser. No. 11/754,569, filed May 29, 2007, now pending, which is a continuation of International application No. PCT/FR2005/003,263, filed Dec. 23, 2005, which is incorporated herein by reference in its entirety; which claims the benefit of priority of French Patent Application No. 04/13,937, filed Dec. 27, 2004.

### BACKGROUND OF THE INVENTION

1. Field of the Invention

The present invention relates to formulations of azetidine derivatives which can be injected or administered orally.

2. Description of the Art

The azetidine derivatives used in the pharmaceutical compositions according to the invention can be denoted by the general formula (Ia) or (Ib) below:

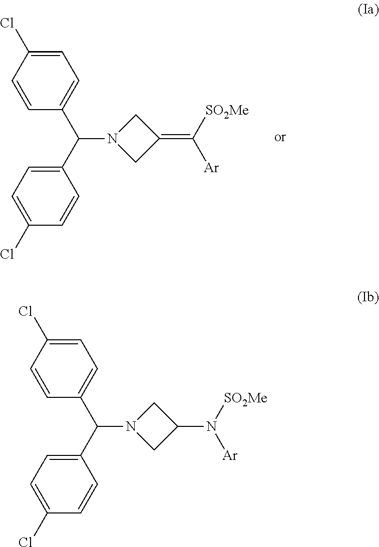

in which Ar is an aromatic or heteroaromatic group optionally substituted by one or more (C₁-C₄)alkyl, halogen, NO₂, CN, (C₁-C₄)alkoxy or OH groups.

In the definition of azetidine derivatives above, the term “aromatic group” is understood to mean in particular a phenyl or naphthyl group, the term “heteroaromatic group” is understood to mean in particular a pyridyl, furyl, thienyl, thiazolyl, imidazolyl or oxazolyl group and the term “halogen” is understood to mean in particular fluorine, chlorine, bromine or iodine.

The product N-{1-[bis(4-chlorophenyl)methyl]azetidin-3-yl}-N-(3,5-difluorophenyl)methylsulfonamide is a specific product of formula (Ib) and corresponds to the specific formula (Ic)

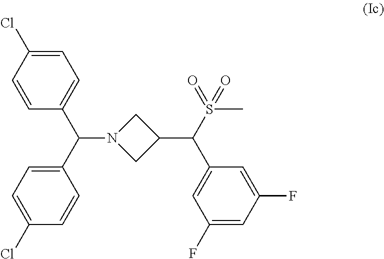

Azetidine derivatives of general formula (Ia) or (Ib) have been disclosed in Patent Applications WO 00/15609, WO 01/64632, WO 01/64633 and WO 01/64634, all of which are incorporated herein by reference in their entirety. In particular, these azetidine derivatives are particularly advantageous for their high affinity for cannabinoid receptors and particularly receptors of the CB1 type.

Unfortunately, azetidine derivatives are products which have very little solubility in water.

Until now, the administration of the azetidine derivatives of general formula (Ia) or (Ib), in particular orally, was envisaged in the form of tablets in formulations comprising, inter alia, cellulose, lactose and other excipients. However, such formulations are still not sufficiently well suited to these products which have little solubility in water due to an excessively low bioavailability.

Numerous documents describe systems capable of dissolving and/or of improving the bioavailability of hydrophobic active principles. However, the systems tested have until now proven to be ineffective in the preparation of pharmaceutical compositions comprising azetidine derivatives defined above which are stable and bioavailable and in which the azetidine derivative is dissolved at an effective concentration.

In particular, J. Pharm. Sciences, 89(8), 967 (2000), and Pharmaceutical Technology Europe, p. 20, September 2000, both of which are incorporated herein by reference in their entirety, mention the formulation of active principles which are not very soluble in water in medium-chain triglycerides. However, the tests carried out with formulations based on Miglyol® have given results which are unsatisfactory from the viewpoint of their bioavailability.

Moreover, International Application WO 95/24893, which is incorporated by reference in its entirety, discloses compositions comprising a digestible oil, a lipophilic surfactant and a hydrophilic surfactant which are intended for the formulation of hydrophobic active principles and for improving their bioavailability. Unfortunately, the above azetidine derivatives have been shown to have an excessively low bioavailability in this type of formulation. In particular, the formulation of such azetidine derivatives in a Miglyol®/Capryol®/Cremophor® system has also been shown to be unsatisfactory in vivo from the pharmacokinetic viewpoint.

As the product has very little solubility, it is also very difficult to envisage an iv formulation or a formulation in the oral and liquid form.

### SUMMARY OF THE INVENTION

It has now been found, and it is this which forms the subject-matter of the present invention, that it is possible to prepare chemically and physically stable pharmaceutical compositions comprising a derivative of general formula (Ia), (Ib) and more particularly (Ic) which make possible delivery of the product in the liquid form which can be administered in the iv form or orally, in particular by drinking.

### DETAILED DESCRIPTION OF THE INVENTION

The present invention relates to formulations composed either of a binary system or of a ternary system which can be injected or administered orally for man.

The present invention relates to a binary system composed of the active principle of formula (Ia) or (Ib) and of the excipient, Polysorbate 80 (POE (polyoxyethylene) monooleate) or Solutol® HS 15 (PEG (polyethylene glycol) hydroxystearate), optionally a cosolvent chosen from ethanol, PEG 400 or propylene glycol.

More particularly, the present invention relates to a binary system composed of the active principle N-{1-[bis(4-chlorophenyl)methyl]azetidin-3-yl}-N-(3,5-difluorophenyl)methylsulfonamide and of the excipient, Polysorbate 80 (POE monooleate) or Solutol® HS 15 (PEG hydroxystearate).

The present invention also relates to a ternary system composed of the active principle of formula (Ia) or (Ib), of the surfactant, Polysorbate 80 (POE monooleate) or Solutol HS 15 (PEG hydroxystearate), and of the cosolvent, ethanol, PEG 400 or propylene glycol. More particularly, the present invention relates to a ternary system composed of the active principle N-{1-[bis(4-chlorophenyl)methyl]azetidin-3-yl}-N-(3,5-difluorophenyl)methylsulfonamide, of the surfactant, Polysorbate 80 (POE monooleate) or Solutol® HS 15 (PEG hydroxystearate), and of the cosolvent, ethanol, PEG 400 or propylene glycol.

The physicochemical characterization of these formulations has demonstrated their ability to dissolve N-{1-[bis(4-chlorophenyl)methyl]azetidin-3-yl}-N-(3,5-difluorophenyl)methylsulfonamide in an aqueous medium up to 3 mg/ml, in comparison with a solubility of N-{1-[bis(4-chlorophenyl)methyl]azetidin-3-yl}-N-(3,5-di-fluorophenyl)methylsulfonamide in water of less than 0.2 μg/ml.

According to the invention, the active principle of general formula (Ia) or (Ib) represents from about 0.01 to about 60% by weight of the total composition. Preferably, it represents from about 0.1 to about 20% by weight and more particularly still from about 0.1% to about 5% by weight of the total composition. For a formulation for the iv route, in which the active principle is completely dissolved and solubilized in a simulated physiological medium, the said active principle represents at most about 5% of the total composition. For a formulation which can be administered orally, the active principle can be in the dispersed state and can represent up to about 60% by weight of the total composition.

According to the invention, the said cosolvent represents from about 1 to about 70% with respect to the total weight of the pharmaceutical composition. Preferably, it represents from about 10 to about 50% by weight and more particularly still from about 20 to about 40% by weight of the total composition.

It is understood that the dosage can vary according to the degree or the nature of the condition to be treated, Thus, the amount of active product in a composition according to the invention will be determined so that a suitable dosage can be prescribed. For this reason, the amount of azetidine derivative of general formula (Ia) or (Ib) varies according to its solubility in the mixture and also according to the dosage appropriate for the treatment of the patients.

In man, the daily doses administered orally are generally between from about 0.1 and about 100 mg of the azetidine derivative of general formula (Ia) or (Ib).

It is understood that, in order to choose the most appropriate dosage, the weight of the patient, his general state of health, his age and all the factors which may influence the effectiveness of the treatment should be taken into account. Preferably, the compositions are prepared so that a unit dose comprises from about 0.1 to about 100 mg of active product.

According to the invention, the active principle of formula (Ia) or (Ib) is dispersed in the surfactant or in a surfactant/cosolvent mixture. In the case of Solutol HS 15 (solid at ambient temperature), the excipient will be melted beforehand at 40-50° C. and subsequently mixed with a cosolvent or directly with the active principle. The combined mixture is kept stirred mechanically until completely homogeneous. Various dosages can be prepared, according to the active principle/excipient(s) starting ratio. For an injectable use, the dosage of active principle cannot be greater than the value of the solubility of the active principle in the excipient or in the excipient/cosolvent mixture.

The following examples, given without implied limitation, illustrate compositions according to the present invention.

### Example 1

Binary system with Solutol HS 15: the active principle (20 mg/g of excipient) is dispersed in the Solutol HS 15 and then kept stirred mechanically until completely dissolved. The Solutol® HS 15 (solid at ambient temperature) was melted beforehand at 40-50° C. The final formulation (concentrate) is solid at ambient temperature and has to be melted before dilution with an isotonic medium and administration by the iv route. The solid formulation (concentrate) is chemically stable at 5° C. for at least 6 months. The dilute formulation (ready-for-use) is chemically and physically stable for at least 6 hours after dilution with an isotonic medium (5% glucose).

### Example 2

Binary system with Polysorbate 80: the active principle (10 mg/g of excipient) is dispersed in the Polysorbate 80 and then kept stirred mechanically until completely dissolved. The Polysorbate was heated beforehand to 40° C. in order to reduce its viscosity. The final formulation (concentrate) is liquid but viscous at ambient temperature. The dilute formulation (ready-for-use) is physically stable for at least 6 hours after dilution with an isotonic medium (5% glucose).

### Example 3

Ternary system with Solutol® HS 15/20% ethanol: the active principle (10 mg/g of excipient) is dispersed in the Solutol HS 15/ethanol 80:20 (w/w) mixture and then kept stirred mechanically until completely dissolved. The Solutol HS 15 (solid at ambient temperature) was melted beforehand at 40-50° C. The final formulation (concentrate) is liquid at ambient temperature and chemically stable at 5° C. for at least 8 months. The dilute formulation (ready-for-use) is chemically and physically stable for at least 24 hours after dilution with an isotonic medium (5% glucose).

### Example 4

Ternary system with Solutol® HS 15/30% propylene glycol: the active principle (10 mg/g of excipient) is dispersed in the Solutol HS 15/propylene glycol 70:30 (w/w) mixture and then kept stirred mechanically until completely dissolved. The Solutol HS 15 (solid at ambient temperature) was melted beforehand at 40-50° C. The final formulation (concentrate) is liquid at ambient temperature and chemically stable at 5° C. for at least 8 months. The dilute formulation (ready-for-use) is chemically and physically stable for at least 24 hours after dilution with an isotonic medium (5% glucose).

What is claimed is:

1. An injectable pharmaceutical composition comprising a compound of formula (Ia) or (Ib) as an active principle: 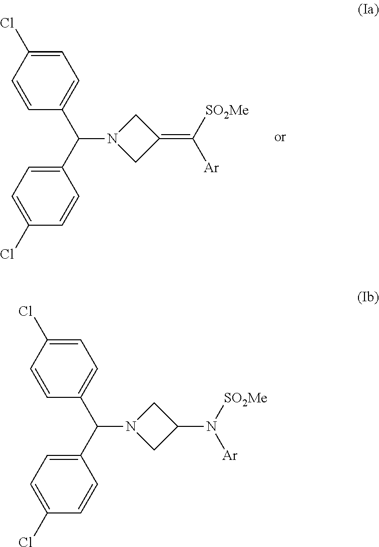
wherein Ar is an aromatic or heteroaromatic group optionally substituted by one or more groups selected from the group consisting of (C₁-C₄)alkyl, halogen, NO₂, CN, (C₁-C₄)alkoxy and hydroxy; and
an excipient selected from Polysorbate 80 (POE monooleate) or SOLUTOL® HS 15 (PEG hydroxystearate); and
optionally a cosolvent chosen from ethanol, PEG 400 or propylene glycol.

2. The pharmaceutical composition according to claim 1, wherein the active principle is present in a proportion of from about 0.01 to about 5% with respect to the total weight of the pharmaceutical composition.

3. The pharmaceutical composition according to claim 1, wherein the active principle is present in a proportion of from about 0.1 to about 4% with respect to the total weight of the pharmaceutical composition.

4. The pharmaceutical composition according to claim 1, wherein the cosolvent is present in a proportion of from about 1% to about 70% with respect to the total weight of the pharmaceutical composition.

5. The pharmaceutical composition according to claim 1, wherein the cosolvent is present in a proportion of from about 20% to about 40% with respect to the total weight of the pharmaceutical composition.

6. The pharmaceutical composition according to claim 1, wherein the active principle is compound of formula (Ib).

7. The pharmaceutical composition according to claim 1, wherein the excipient is Polysorbate 80 (POE monooleate).

8. The pharmaceutical composition according to claim 1, wherein the excipient is SOLUTOL® HS 15 (PEG hydroxystearate).

9. The pharmaceutical composition according to claim 1, wherein the cosolvent is ethanol.

10. The pharmaceutical composition according to claim 1, wherein the cosolvent is PEG 400.

11. The pharmaceutical composition according to claim 1, wherein the cosolvent is propylene glycol.

12. An injectable pharmaceutical composition comprising an active principle, N-{1-[bis(4-chlorophenyl)methyl]-azetidin-3-yl}-N-(3,5-difluorophenyl)methylsulfonamide, and an excipient, Polysorbate 80 (POE monooleate) or SOLUTOL® HS 15 (PEG hydroxystearate).

13. The pharmaceutical composition according to claim 12, wherein the active principle is present in a proportion of from about 0.01 to about 5% with respect to the total weight of the pharmaceutical composition.

14. The pharmaceutical composition according to claim 12, wherein the active principle is present in a proportion of from about 0.1 to about 4% with respect to the total weight of the pharmaceutical composition.

15. The pharmaceutical composition according to claim 12, wherein the excipient is Polysorbate 80 (POE monooleate).

16. The pharmaceutical composition according to claim 12, wherein the excipient is SOLUTOL® HS 15 (PEG hydroxystearate).

17. An injectable pharmaceutical composition comprising an active principle, N-{1-[bis(4-chlorophenyl)methyl]azetidin-3-yl}-N-(3,5-di-fluorophenyl)methylsulfonamide, a surfactant, Polysorbate 80 (POE monooleate) or SOLUTOL® HS 15 (PEG hydroxystearate), and a cosolvent chosen from ethanol, PEG 400 or propylene glycol.

18. The pharmaceutical composition according to claim 17, wherein the active principle is present in a proportion of from about 0.01 to about 5% with respect to the total weight of the pharmaceutical composition.

19. The pharmaceutical composition according to claim 17, wherein the active principle is present in a proportion of from about 0.1 to about 4% with respect to the total weight of the pharmaceutical composition.

20. The pharmaceutical composition according to claim 17, wherein the cosolvent is present in a proportion of from about 1% to about 70% with respect to the total weight of the pharmaceutical composition.

21. The pharmaceutical composition according to claim 17, wherein the cosolvent is present in a proportion of from about 20% to about 40% with respect to the total weight of the pharmaceutical composition.

22. The pharmaceutical composition according to claim 17, wherein the surfactant is Polysorbate 80 (POE monooleate).

23. The pharmaceutical composition according to claim 17, wherein the surfactant is SOLUTOL® HS 15 (PEG hydroxystearate).

24. The pharmaceutical composition according to claim 17, wherein the cosolvent is ethanol.

25. The pharmaceutical composition according to claim 17, wherein the cosolvent is PEG 400.

26. The pharmaceutical composition according to claim 17, wherein the cosolvent is propylene glycol.

27. The pharmaceutical composition according to claim 18, wherein the surfactant is SOLUTOL® HS 15 (PEG hydroxystearate) and the cosolvent is ethanol and said SOLUTOL® and ethanol are present in a proportion of about 80/20 (w/w) with 5% of glucose.

28. The pharmaceutical composition according to claim 18, wherein the surfactant is SOLUTOL® HS 15 (PEG hydroxystearate) and the cosolvent is propylene glycol and said SOLUTOL® and propylene glycol are present in a proportion of about 70/30 (w/w) with 5% of glucose.
